(19) United States
(12) Patent Application Publication
(10) Pub. No.: US 2024/0390535 A1
(43) Pub. Date: Nov. 28, 2024

(54) ULTRAVIOLET LIGHT IRRADIATION SHEET

(71) Applicant: NIPPON TELEGRAPH AND TELEPHONE CORPORATION, Tokyo (JP)

(72) Inventors: Asuka MIYAKE, Musashino-shi, Tokyo (JP); Hitoshi SESHIMO, Musashino-shi, Tokyo (JP); Ryuji YAMAMOTO, Musashino-shi, Tokyo (JP); Tomohiro TANIGUCHI, Musashino-shi, Tokyo (JP); Ayako IWAKI, Musashino-shi, Tokyo (JP); Satoshi NARIKAWA, Musashino-shi, Tokyo (JP); Takahito KIRIHARA, Musashino-shi, Tokyo (JP); Kazuhide NAKAJIMA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP); Nobutomo HANZAWA, Musashino-shi, Tokyo (JP)

(73) Assignee: NIPPON TELEGRAPH AND TELEPHONE CORPORATION, Tokyo (JP)

(21) Appl. No.: 18/695,575

(22) PCT Filed: Sep. 30, 2021
(86) PCT No.: PCT/JP2021/036073

§ 371 Date: Mar. 26, 2024

(51) Int. Cl. A61L 2/10; A61L 2/26; G02B 6/10

(52) U.S. Cl. CPC A61L 2/10; A61L 2/26; G02B 6/102; A61L 2202/11

(57) ABSTRACT

An object of the present invention is to provide an ultraviolet light irradiation sheet that can be decontaminated regardless of a shape of an object to be decontaminated. An ultraviolet light irradiation sheet (50) includes an optical fiber (20) which emits a part of propagating light from a side surface or an end surface; and a deformable sheet (10) which incorporates the optical fiber (20). The ultraviolet light irradiation sheet uniformly or partially irradiates ultraviolet light from a sheet surface. Therefore, by covering the object to be sterilized with the ultraviolet light irradiation sheet, a material having a complicated shape or an amorphous shape can be irradiated with the ultraviolet light without making a shade. Since the ultraviolet light irradiation sheet does not diffuse bacteria and viruses by wind, the sterilization of the object to be sterilized can be performed without forming a closed space.

### TECHNICAL FIELD

The present disclosure relates to an ultraviolet light irradiation sheet which performs sterilization and inactivation of viruses, using ultraviolet light.

### BACKGROUND ART

For the purpose of preventing infectious diseases or the like, there is an increasing demand for systems which performs sterilization and inactivation of viruses using ultraviolet light. In the present embodiment, “decontamination” is assumed to include sterilization and inactivation of viruses.

In the case of sterilizing the surface of an object to be sterilized by irradiating with ultraviolet light, the object is generally irradiated with an ultraviolet light using ultraviolet light, but depending on the shape of the object, the object may not be exposed to light due to being shaded.

For example, a device for removing bacteria and viruses that are submerged in portions that are not directly irradiated with ultraviolet light by applying an airflow to an object having an uneven surface such as a carpet to move bacteria and viruses has been proposed (see, for example, PTL 1)

### CITATION LIST

### Patent Literature

[PTL 1] Japanese Patent Application Publication No. 2019-216831

### SUMMARY OF INVENTION

### Technical Problem

However, the device disclosed in PTL 1 needs to have the object to be sterilized confined in an enclosed space so that the transferred bacteria and viruses are not spread. In this case, it is necessary that the object be smaller than the irradiation unit or that the object have a flat shape to allow close contact with the irradiation part to form an enclosed space. In other words, the device disclosed in PTL 1 has a problem that the shape of the object to be sterilized is limited.

In order to solve the above problems, an object of the present invention is to provide an ultraviolet light irradiation sheet that can sterilize regardless of the shape of an object to be sterilized.

### Solution to Problem

In order to achieve the above object, the ultraviolet light irradiation sheet according to the present invention is in the form of a sheet, and has a structure capable of radiating ultraviolet light from the sheet surface.

Specifically, the ultraviolet light irradiation sheet according to the present invention includes an optical fiber which emits a supplied ultraviolet light, and a deformable sheet which incorporates the optical fiber.

The ultraviolet light irradiation sheet radiates the ultraviolet light from the sheet surface, by the optical fiber emitting the supplied ultraviolet light from a side surface or a tip. Therefore, by covering the object to be sterilized with the ultraviolet light irradiation sheet, a material having a complicated shape or an amorphous shape can be irradiated with the ultraviolet light without being shaded. Since the ultraviolet light irradiation sheet does not diffuse bacteria and viruses by wind, the sterilization of the object to be sterilized can be performed without forming a closed space.

Accordingly, the present invention can provide the ultraviolet light irradiation sheet capable of sterilizing regardless of the shape of the object to be sterilized.

The sheet of the ultraviolet light irradiation sheet according to the present invention may be a fiber fabric, and the optical fiber may be woven into the fiber.

The sheet of the ultraviolet light irradiation sheet according to the present invention may be a resin, and the optical fiber may be embedded in the resin.

The sheet of the ultraviolet light irradiation sheet according to the present invention is characterized in that one surface is shielded from light. The ultraviolet light can be prevented from being radiated to a part other than the object to be sterilized.

The above inventions can be combined as far as possible.

### Advantageous Effects of Invention

The present invention can provide an ultraviolet light irradiation sheet that can sterilize regardless of the shape of an object to be sterilized.

### BRIEF DESCRIPTION OF DRAWINGS

FIG. 1 is a drawing for explaining an ultraviolet light irradiation sheet according to the present invention.

FIG. 2 is a diagram for explaining a cross section of the ultraviolet light irradiation sheet according to the present invention.

FIG. 3 is a diagram for explaining a cross section of the ultraviolet light irradiation sheet according to the present invention.

FIG. 4 is a diagram for explaining a usage method of the ultraviolet light irradiation sheet according to the present invention.

FIG. 5 is a diagram for explaining the ultraviolet light irradiation sheet according to the present invention.

FIG. 6 is a diagram for explaining the ultraviolet light irradiation sheet according to the present invention.

FIG. 7 is a diagram for explaining a cross section of the ultraviolet light irradiation sheet according to the present invention.

FIG. 8 is a diagram for explaining a cross section of the ultraviolet light irradiation sheet according to the present invention.

FIG. 9 is a diagram for explaining a cross section of the ultraviolet light irradiation sheet according to the present invention.

### DESCRIPTION OF EMBODIMENTS

Embodiments of the present invention will be described with reference to the accompanying drawings. The embodiments described below are examples of the present invention, and the present invention is not limited to the following embodiments. Note that, in the present specification and the drawings, components having the same reference numerals indicate the same components.

### Embodiment 1

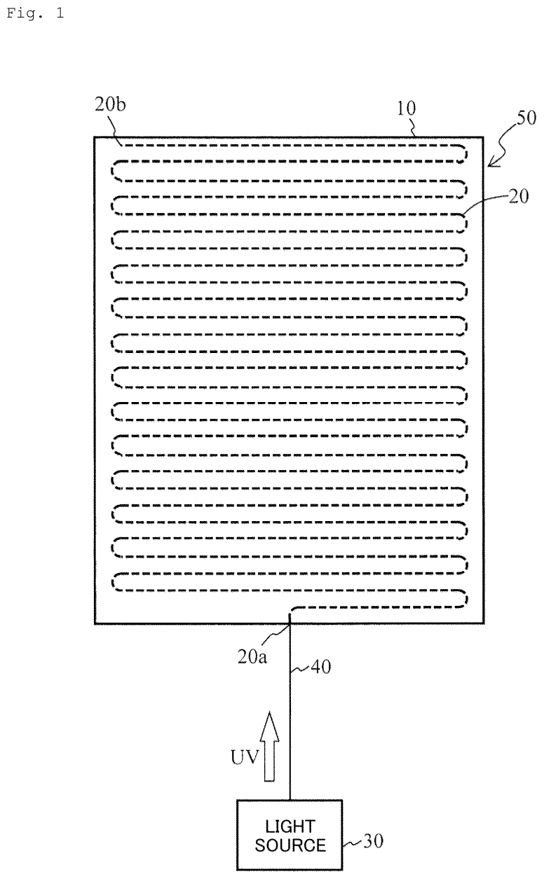

FIG. 1 is a diagram for explaining an ultraviolet light irradiation sheet 50 of the present embodiment. The ultraviolet light irradiation sheet 50 includes an optical fiber 20 for emitting a part of propagating light from a side surface, and a deformable sheet 10 that incorporates the optical fiber 20.

The optical fiber 20 is an optical fiber that allows propagating light to leak from the side surfaces. For example, the optical fiber 20 includes a scattering body in a core, and light propagating through the core is scattered by the scattering body, and the scattered light is emitted from a side surface via a clad.

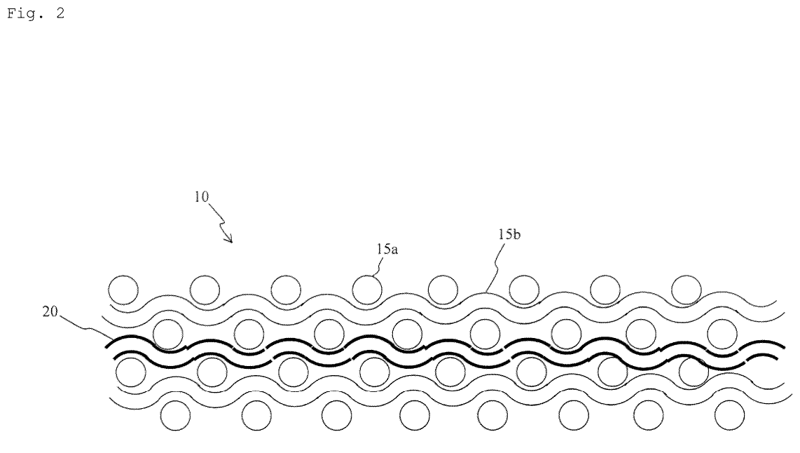

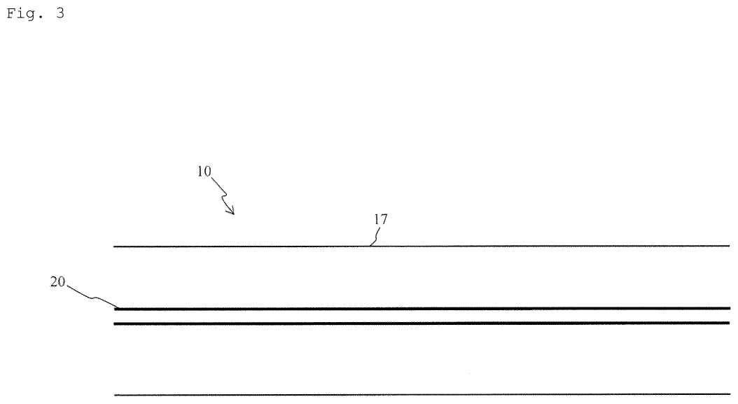

The sheet 10 incorporates the optical fiber 20 and can be freely folded and rolled. For example, as shown in FIG. 2, the sheet 10 is a fabric made of fibers 15, and the optical fibers 20 may be woven into the fibers 15. As shown in FIG. 3, the sheet 10 is made of resin 17, and the optical fiber 20 may be embedded in the resin 17.

It is preferable that the optical fibers 20 be disposed in the sheet 10 in a zigzag as shown in FIG. 1. Light can be emitted from the entire surface of the sheet 10. In the case where the optical fibers 20 are disposed in zigzag as shown in FIG. 1, the radius of curvature is set to such an extent that the optical fibers 20 are not damaged at the folded part.

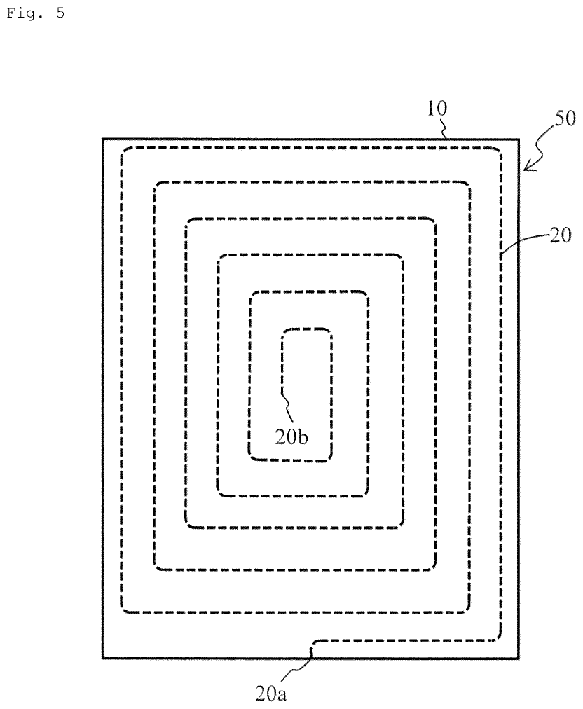

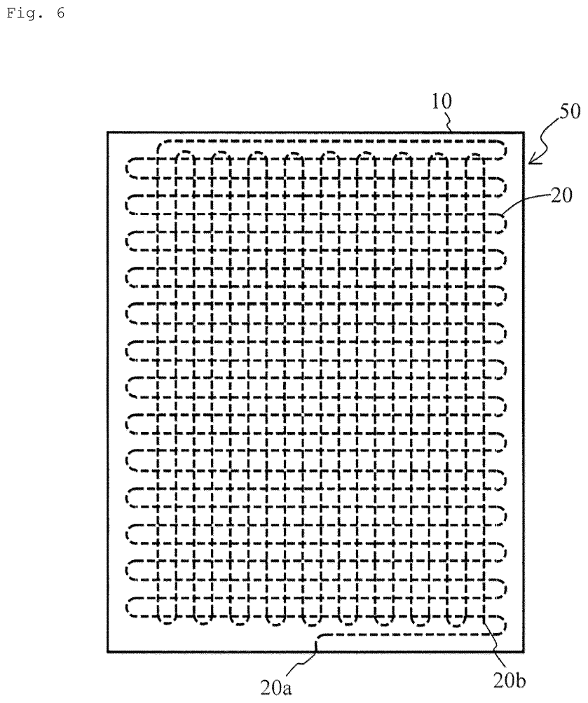

FIGS. 5 and 6 are diagrams for explaining other placements of the optical fibers 20 inside the sheet 10. In FIG. 5, the optical fibers 20 are disposed spirally with respect to the sheet surface. In FIG. 6, the optical fibers 20 are disposed in a lattice shape with respect to the sheet surface. Any placement can be realized by one optical fiber. Also, in any placement, the radius of curvature is set to such an extent that the optical fiber 20 is not damaged at the folded part.

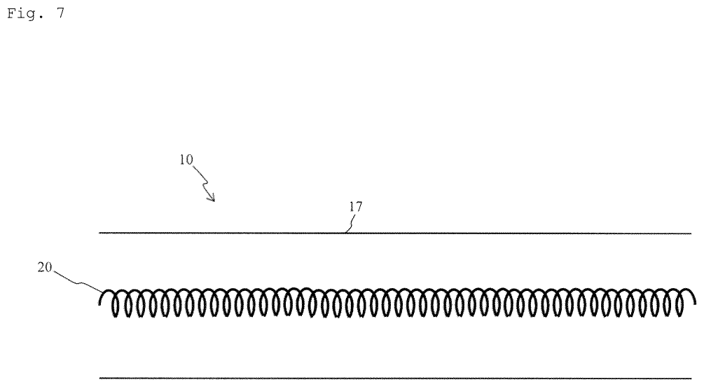

The optical fibers disposed as shown in FIGS. 1, 5 and 6 are not limited to the optical fibers 20 knitted into the fibers 15 as shown in FIG. 2 and the optical fibers 20 may be linearly embedded in the resin 17 as shown in FIG. 3. For example, the optical fiber 20 may be spirally embedded in the resin 17 as shown in FIG. 7.

One end of the optical fiber 20 is installed as a connection end 20a in a part of the sheet 10. A light source 30 disposed outside the ultraviolet light irradiation sheet 50 and the connection end 20a are connected by an optical fiber 40. The ultraviolet light output from the light source 30 is supplied to the connection end 20a via the optical fiber 40 and propagated inside the optical fiber 20. The ultraviolet light leaks from the side surface of the optical fiber 20 during propagation in the optical fiber 20, and is radiated to the outside from the whole surface of the sheet 10. The ultraviolet light may be radiated not only from the side surface of the optical fiber 20 but also from the end portion 20b on the opposite side to the connection end 20a.

It is preferable that one surface of the sheet 10 be shielded from light. When it is not shielded, ultraviolet light is emitted from both surfaces of the sheet 10, but when one surface is shielded (light-shielding surface), ultraviolet light is emitted from only the other surface (radiation surface). As will be described later, if the ultraviolet light irradiation sheet 50 is disposed with the radiation surface directed toward the object to be sterilized, only the object to be sterilized is irradiated with ultraviolet light, and ultraviolet light can be prevented from being irradiated to objects other than the object to be sterilized such as surrounding people and animals.

### Embodiment 2

In embodiment 1, an example in which ultraviolet light is emitted from the side surface of the optical fiber was described. In this embodiment, an example in which ultraviolet light is emitted from a tip (end surface) of the optical fiber will be described.

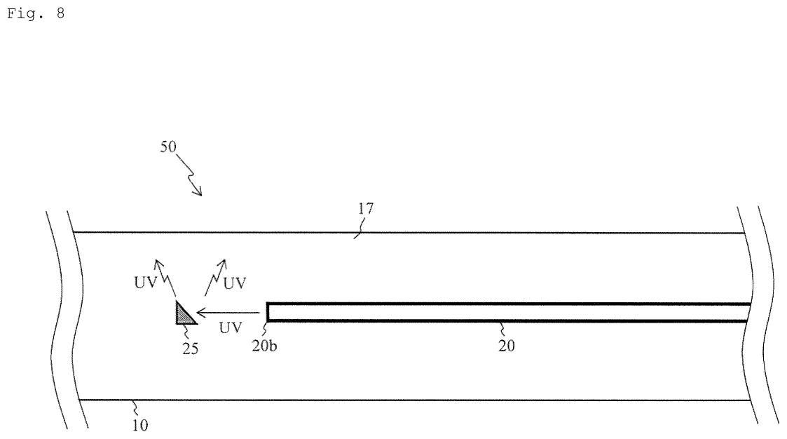

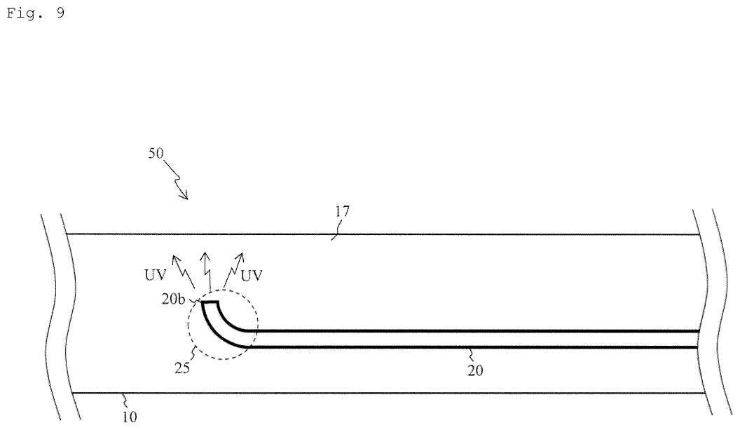

The ultraviolet light irradiation sheet 50 may have a structure as shown in FIG. 8 or 9. An optical fiber 20 of the ultraviolet light irradiation sheet 50 of FIG. 8 or FIG. 9 emits propagating ultraviolet light from the end portion 20b, is embedded in the sheet 10, and further has a turning function 25 which directs the ultraviolet light emitted from the end portion 20b of the optical fiber toward one surface (upward in FIGS. 8 and 9) of the sheet 10.

The turning function 25 is, for example, a prism for scattering the ultraviolet light in a plurality of directions as shown in FIG. 8. Alternatively, the turning function 25 may be a shaped portion in which the end portion 20b of the optical fiber 20 is bent perpendicularly to the surface of the sheet 10 and ultraviolet light can be radiated perpendicularly to the surface of the sheet 10, as shown in FIG. 9. In this case, it is preferable that the end portion 20b be processed into a shape (for example, unevenness processing) for scattering the ultraviolet light in a plurality of directions. The ultraviolet light irradiation sheet 50 shown in FIGS. 8 and 9 does not require the light shielding and reflecting surfaces described above or described later due to the turning function 25.

In the structure of the ultraviolet light irradiation sheet 50 of the present embodiment, there is a possibility that the intensity of the ultraviolet light which can be irradiated from the surface of the sheet 10 is uneven. The ultraviolet light irradiation sheet 50 of the present embodiment can partially enhance the sterilization effect by utilizing the intensity unevenness.

The ultraviolet light irradiation sheet 50 may have a structure in which the structure of embodiment 1 and the structure of embodiment 2 are combined (a structure in which ultraviolet light is emitted from the side surface of the optical fiber 20 and ultraviolet light is also emitted from the end portion 20b of the optical fiber 20).

### Examples

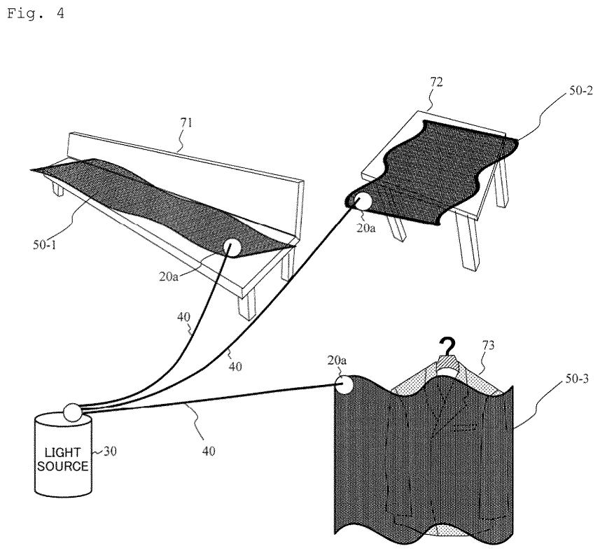

FIG. 4 is a diagram for explaining a sterilization system including the ultraviolet light irradiation sheet 50 In FIG. 1, although a case where the ratio between the light sources 30 and the ultraviolet light irradiation sheets 50 is 1:1 has been described, as shown in FIG. 4, by branching the ultraviolet light output from one light source 30 into a plurality of portions, it is possible to sterilize a plurality of portions, using a plurality of ultraviolet light irradiation sheets 50.

One of the parts of branched ultraviolet light is input to an ultraviolet light irradiation sheet 50-1. The ultraviolet light irradiation sheet 50-1 is disposed to cover a seat surface of a bench 71. Here, if one surface of the ultraviolet light irradiation sheet 50-1 is a light-shielding surface, the irradiation surface is disposed toward the seat surface of the bench 71. The ultraviolet light from the light source 30 is irradiated from the irradiation surface of the ultraviolet light irradiation sheet 50-1, and the seat surface of the bench 71 is sterilized.

One of the branched ultraviolet light is input to an ultraviolet light irradiation sheet 50-2. The ultraviolet light irradiation sheet 50-2 is disposed to cover a top plate of a table 72. When one surface of the ultraviolet light irradiation sheet 50-2 is a light-shielding surface, the irradiation surface is disposed toward the top plate of the table 72. The ultraviolet light from the light source 30 is irradiated from the irradiation surface of the ultraviolet light irradiation sheet 50-2, and the top plate of the table 72 is sterilized.

One of the branched ultraviolet light is input to an ultraviolet light irradiation sheet 50-3. The ultraviolet light irradiation sheet 50-3 is disposed to cover clothing 73 hung on the hanger. Here, when one surface of the ultraviolet light irradiation sheet 50-3 is a light-shielding surface, the irradiation surface is disposed toward the clothing 73. The ultraviolet light from the light source 30 is radiated from the irradiation surface of the ultraviolet light irradiation sheet 50-3, and the clothing 73 is sterilized.

Since the ultraviolet light irradiation sheet 50 is freely deformable in this way, the ultraviolet light irradiation sheet 50 can be brought into close contact in accordance with the shape of the object to be sterilized. Therefore, the ultraviolet light irradiation sheet 50 can uniformly or partially irradiate an object to be sterilized having a complicated shape or an object to be sterilized having an irregular shape with ultraviolet light without shading being formed. Therefore, it is not necessary to move bacteria and viruses by wind, and since the bacteria and viruses do not diffuse, it is not necessary to create a closed space between the irradiation part and the object to be sterilized. Accordingly, the present invention can provide the ultraviolet light irradiation sheet capable of being decontaminated regardless of the shape of the object to be decontaminated.

### Reference Signs List

10 Sheet
15 Fiber (15a is vertical yarn and 15b is horizontal yarn)
17 Resin
20 Optical fiber
20a Connection end
20b End portion
25 Turning function
30 Light source
40 Optical fiber
50 Ultraviolet light irradiation sheet

1. An ultraviolet light irradiation sheet comprising:
an optical fiber which emits a supplied ultraviolet light; and
a deformable sheet which incorporates the optical fiber.

2. The ultraviolet light irradiation sheet according to claim 1, wherein the sheet is a fiber fabric, and the optical fiber is woven into the fiber.

3. The ultraviolet light irradiation sheet according to claim 1, wherein the sheet is a resin, and the optical fiber is embedded in the resin.

4. The ultraviolet light irradiation sheet according to claim 1, wherein the sheet has one surface shielded from light.
